(19) United States
(12) Patent Application Publication
(10) Pub. No.: US 2025/0205239 A1
(43) Pub. Date: Jun. 26, 2025

(54) COMPOSITIONS AND METHODS FOR PREVENTING AND TREATING NEURODEGENERATIVE DISEASES

(71) Applicant: ARIBIO CO., LTD., Gyeonggi-do (KR)

(72) Inventors: Jai Jun Choung, Gyeonggi-do (KR); Bo Seung Seo, Gyeonggi-do (KR); Byungwoo Kang, Busan (KR)

(21) Appl. No.: 18/991,922

(22) Filed: Dec. 23, 2024

Related U.S. Application Data
(63) Continuation of application No. PCT/IB2023/056532, filed on Jun. 24, 2023.
(60) Provisional application No. 63/367,033, filed on Jun. 24, 2022.

(51) Int. Cl. A61K 31/519; A61K 31/325; A61K 31/445; A61K 31/55; A61P 25/28

(52) U.S. Cl. CPC A61K 31/519; A61K 31/325; A61K 31/445; A61K 31/55; A61P 25/28

(57) ABSTRACT

The present invention provides a composition for preventing or treating a neurodegenerative disease containing a phosphodiesterase 5 inhibitor (PDE5 inhibitors) and an acetylcholinesterase inhibitor (AChEI) and a method using thereof, wherein the PDE5 inhibitor is selected from among mirodenafil, sildenafil, vardenafil, tadalafil, udenafil, dasantafil, avanafil, pharmaceutically acceptable salts, solvates, hydrates, and a mixture thereof; and the AchEI is selected from among donepezil, rivastigmine, galantamine, physostigmine, tacrine, metrifonate, phenserine, tolserine, eseroline, huperizine A and B, galangin, cardanol, donepezil-AP2238, donepezil-tacrine, tacrine-ferulic acid hybrid, tacrine-hydroxyquinoline, ladostigil, indenyl derivatives, pharmaceutically acceptable salts, solvates, hydrates and a mixture thereof; and the neurodegenerative disease is dementia, Parkinson's disease (PD), Alzheimer's disease (AD), Huntington's disease (HD) or Multiple sclerosis (MS).

### CROSS-REFERENCE TO RELATED APPLICATIONS

This application claims the benefit of priority from U.S. Provisional Application Ser. No. 63/367,033, filed Jun. 24, 2022, the contents of which are incorporated herein by reference.

### FIELD OF THE DISCLOSURE

The present invention relates to a composition containing a phosphodiesterase 5 inhibitor (PDE5 inhibitors) and an acetylcholinesterase inhibitor (AChEI) for preventing or treating neurodegenerative diseases and a method using thereof.

### SEQUENCE LISTING

This application incorporates by reference in its entirety the Sequence Listing XML file entitled “04334900119_SequenceListing.xml (7 KB)”, which was created on Jun. 23, 2023, and filed electronically herewith.

### BACKGROUND OF THE DISCLOSURE

Recently, patients with degenerative neurological disorders have been increased rapidly. In the treatment of degenerative neurological disorder, the most important step is the prevention. However, the cause of the disease has not been clearly understood yet and thus a treatment method is still needed to be studied. The common pathological phenomenon of degenerative neurological disorders is the death of central nervous system cells. Unlike other organ cells, central nervous system cells are almost impossible to regenerate after cell-death, resulting in permanent loss of function. Thus, the methods for the treatment of such brain diseases developed so far are mainly focused on the analysis of the death mechanism of nerve cells themselves and the prevention of the death based on the analysis.

Neurodegeneration, as a collective term, involves the progressive loss of structure or function of neurons, including death of neurons in various areas of the Brain. Neurodegenerative diseases including dementia, Parkinson's disease (PD), Alzheimer's disease (AD), Huntington's disease (HD) and Multiple sclerosis (MS) are emerging as a serious challenge to the ageing population. A potential causes for neurodegeneration or neuronal cell death are oxidative stress, increased protein such as alpha- or beta-amyloid) aggregates in the neurons and chronic inflammation in the Central Nervous System (CNS).

Recent studies on Alzheimer's disease and Parkinson's disease, for example, provide that inflammatory reaction in the brain is a major cause of neuronal death. In reality, the increase of inflammation mediators and reactive oxygen has been confirmed in the cerebrospinal fluid of brain disease patients. Also, numbers of active microglial cells are observed in the area of brain damage, indicating brain inflammation is a major cause of Parkinson's disease. Therefore, inhibition of brain inflammation by neuroglial cells has become a target of treating degenerative neurological disorder. However, therapeutic agents that have been developed so far are only effective in regulating the symptoms of the disease but are not effective in treating degenerative neurological disorder itself.

Therefore, it is required to develop a preventive and therapeutic agent for degenerative neurological disorder based on the totally different concept from the conventional ones.

For example, dementia is an acquired brain disease with multifaceted pathogenesis caused by various genetic and environmental risk factors and refers to a clinical disease that causes multiple cognitive deficits. The most representative disease causing dementia is Alzheimer's disease that occurs mainly in elderlies and contribute to more than 60% of dementia.

Studies implicate inflammation in neurodegenerative diseases like Parkinson's disease, Alzheimer's disease, Huntington's disease, etc. Contrary to the traditionally held belief that the brain is an immune-privileged site due to the presence of the Blood-Brain Barrier (BBB), recent studies have established that the brain is fully capable of mustering an immune response. Inflammation in the brain does not involve the peripheral immune system and does not involve antibodies or T-Cells. The immune reaction in the brain depends on the synthesis of inflammatory components by Glial cells especially the resident phagocytes, which in the case of the brain, are the microglia.

Within the brain, glial cells play a critical role in maintaining a microenvironment of homeostatis that promotes neuronal survival. Microglia mediate innate immune responses to invading pathogens by secreting a myriad of factors that include, cytokines, chemokines, prostaglandins, reactive oxygen and nitrogen species, and growth factors. Therefore, pro- and anti-inflammatory responses must be tightly regulated to prevent the potential detrimental effects of prolonged inflammation-induced oxidative stress on vulnerable neuronal populations.

In the normal adult brain, microglial cells are usually in the resting state. When activated, these cells are known to release various types of pro-inflammatory molecules such as Nitric Oxide (NO) and cytokines which cause damage and cell death in the surrounding neurons. For example, activated microglia, accumulation of cytokines as well as nuclear factor kappa B (NF-.kappa.B) pathway activation has been found to contribute to the progression of neurodegenerative diseases.

On the other hand, research on amyloid beta protein (Aβ), which is known to be a common cause of hereditary and sporadic Alzheimer's disease, reported that even in normal people, Aβ is produced in small amounts in various parts of the body. In normal people, Aβ is rapidly degraded after being produced and does not accumulate in the body, but in the case of patients with Alzheimer's disease, Aβ is produced in an abnormally large amount and is accumulated in tissues without being degraded, resulting in the generation of senile plaques or excessive accumulation in places such as the hippocampus or cerebral cortex, which play an important role in memory and learning. The accumulated Aβ triggers an inflammatory response in surrounding cells. As a result, nerve cells become damaged and even the neural networks for maintaining the normal function of the brain end up being damaged. Furthermore, the accumulated Aβ produces a great deal of active oxygen that activates the signaling system that kills nerve cells.

Aβ is a part of amyloid precursor protein cleaved by β-secretase. There are various forms of Aβ depending on the number of amino acids constituting it. In the case of patients with Alzheimer's disease, the proportion of Aβ composed of 40 or 42 amino acids increases rapidly. There are many reports that Aβ induces neuronal cell death when treated with nerve cells cultured in vitro and that the mechanism of cell death is similar to the type of apoptosis seen in patients with Alzheimer's disease. Damage to nerve cells by Aβ1-42 or Aβ1-43 protein has been identified as one of the important causes of Alzheimer-type disease, and Aβ25-35 is known to be an important toxic fragment of Aβ1-42 or 43 that causes nerve cell damage.

The most common drugs currently approved by the FDA and used to treat dementia include AChEI and NMDA (N-Methyl-D-aspartate) receptor antagonists, and various other drugs, such as antioxidants, nonsteroidal anti-inflammatory drugs (NSAID), anti-inflammatory agents, statins, and hormones, are used in combination therewith. However, these drugs are only used to relieve and delay symptoms and improve cognitive function, and currently, a fundamental treatment for dementia is still in need.

Representative AChEIs include donepezil, rivastigmine, and galantamine, and these drugs show a symptomatic treatment effect by temporarily increasing the concentration of acetylcholine, which is a neurotransmitter. In addition, these drugs are prescribed to patients with mild to moderate Alzheimer's Disease, vascular dementia, Parkinson's Disease dementia, and stroke or accompanying subcortical ischemic vascular disease.

Due to the decrease or loss of nerve cell function, degenerative neurological diseases including dementia show abnormalities in a wide variety of functions including all functions of the body that can be felt, such as the motor control function, cognitive function, perceptive function, and sensory function of the human body, as well as the autonomic nervous function, which is self-regulated in a state that the human body is not aware of.

Because the cause of the neurodegenerative diseases including dementia is not completely understood, a fundamental treatment is still at challenge. The commercially available drugs can only relieve symptoms in some diseases, but fail to fundamentally change the progression of the disease, and tolerance and serious side effects of the drug emerge after treatment, which further limits the improvement of symptoms in these patients.

Nevertheless, the options for treating neurodegenerative diseases or disorders including dementia are still limited.

### SUMMARY

The present invention provides a composition and a method for treating a neurodegenerative disease by reducing the neuroinflammation especially in CNS system and/or by reducing the expression of a toxic protein such as beta-amyloid (Ab),
wherein,
the composition comprising a PDE-5 inhibitor and an acetylcholinesterase inhibitors (AChEI),
the PDE-5 inhibitor is selected from among mirodenafil, sildenafil, vardenafil, tadalafil, udenafil, dasantafil, avanafil; and pharmaceutically acceptable salts, solvates, hydrates, or a mixture thereof;
the AchEI is selected from among donepezil, galantamine, rivastigmine, or a mixture thereof;
the composition inhibits proinflammatory cytokines such as IL1b, IL-6, or TNFa;
the composition inhibits the growth and differentiation of nerve cells and degenerating learning and memory, to induce a decrease in intracellular Aβ, thereby increasing the protection of nerve cells and synaptic plasticity; and
the neurodegenerative disease is selected from the group among dementia, Parkinson's disease (PD), Demential with Lewy body (DLB), Alzheimer's disease (AD), Huntington's disease (HD), Multiple sclerosis (MS), Vascular Dementia (VaD), or a mixed etiologies thereof.

### BRIEF DESCRIPTION OF FIGURES

FIG. 1 and FIG. 2 show that compositions containing mirodenafil and Donepezil provide synergistic effects on IL1β inhibition, for example, at the ratio of mirodenafil:Donepezil at 5:1, 2:1, 1:1, 1:2, 1:5, or 1:10.

FIG. 3 and FIG. 4 show that compositions containing mirodenafil and Donepezil provide synergistic effects on TNFα inhibition, for example, at the ratio of mirodenafil:Donepezil at 10:1, 5:1, 2:1, 1:1, 1:2, or 1:5.

FIG. 5 and FIG. 6 show that compositions containing mirodenafil and Galantamine provide synergistic effects on IL1β inhibition, for example, at the ratio of mirodenafil:Galantamine at 10:1, 5:1, 2:1, 1:1, 1:2, 1:5, or 1:10.

FIG. 7 and FIG. 8 show that compositions containing mirodenafil and Galantamine provide synergistic effects on IL1β inhibition, for example, at the ratio of mirodenafil:Galantamine at 10:1, 5:1, 2:1, 1:1, 1:2, 1:5, or 1:10.

FIG. 9 and FIG. 10 show that compositions containing mirodenafil and Rivastigmine has synergistic effect on IL1β inhibition, for example, at the ratio of mirodenafil:Rivastigmine at 5:1, 2:1, 1:1, 1:2, or 1:5.

FIG. 11 and FIG. 12 show that compositions containing mirodenafil and Rivastigmine has synergistic effect on TNFα inhibition, for example, at the ratio of mirodenafil:Rivastigmine at 5:1, 2:1, 1:1, 1:2, 1:5, or 1:10.

FIGS. 13-18 provide that mirodenafil (AR1001) and all three of the AChEIs has synergistic effect on reducing Aβ42 accumulation at the ratio of, for example, AR1001:AChEIs at 5:1, 1:1, or 1:5.

FIG. 13 and FIG. 14 show results of the decrease in intracellular Aβ according to the combined treatment of mirodenafil and donepezil according to an embodiment of the present invention.

FIG. 15 and FIG. 16 show synergistic effect on the decrease in intracellular Aβ by the composition of the present invention containing mirodenafil and Galantamine.

FIG. 17 and FIG. 18 show synergistic effect on the decrease in intracellular Aβ by the composition of the present invention containing mirodenafil and rivastigmine.

### DETAILED DESCRIPTION

The present invention provides a composition and a method for treating a neurodegenerative disease by reducing the neuroinflammation especially in CNS system and/or by reducing the expression of a toxic protein such as beta-amyloid (Ab),
wherein,
the composition comprising a PDE-5 inhibitor and an acetylcholinesterase inhibitors (AChEI),
the PDE-5 inhibitor is selected from among mirodenafil, sildenafil, vardenafil, tadalafil, udenafil, dasantafil, avanafil; and pharmaceutically acceptable salts, solvates, hydrates, or a mixture thereof;
the AChEI is selected from among donepezil, galantamine, rivastigmine, or a mixture thereof;
the composition inhibits proinflammatory cytokines such as IL1b, IL-6, or TNFa;
the composition inhibits the growth and differentiation of nerve cells and degenerating learning and memory, to induce a decrease in intracellular Aβ, thereby increasing the protection of nerve cells and synaptic plasticity; and
the neurodegenerative diseases is selected from among dementia, Parkinson's disease (PD), Demential with Lewy body (DLB), Alzheimer's disease (AD), Huntington's disease (HD), Multiple sclerosis (MS), Vascular Dementia (VaD), or a mixed etiologies thereof.

In an embodiment of the present invention provides a composition for preventing and treating dementia comprising a phosphodiesterase 5 inhibitor and an acetylcholinesterase inhibitor as active ingredients.

In a certain embodiment of the present invention, the composition comprises the weight % of PDE-5 inhibitor and an AChEI is from 1:0.1 to 1:10 or 50:1, 10:1, 5:1, 2:1, 1:1, 1:2, 1:5, or 1:10.

In another embodiment, the compositions of the present invention provides a synergistic effect for:
(1) inhibition of Aβ Oligomer/Fibril formation by reduction of Aβ aggregation;
(2) inhibition of β-Amyloidogenic Processing decreased BACE-1;
(3) reduction of extracellular Aβ monomers, oligomers & Aβ Fibril/Plaque by increase of the cerebral blood flow;
(4) suppression of neuronal cell death Inhibition and promotion of neurogenesis, synaptogenesis and/or angiogenesis by activation of NO/cGMP/PKG/CREB Pathway,
(5) restoration of synaptic plasticity (synaptic Plasticity) by activation of Wint Signaling by inhibition of DKK-1, and inhibition of production of APP and reduction of Aβ accumulation by suppression of positive feedback loop for Aβ production, and
(6) inhibition of formation of Aβ Fibril/plaque by removal of intracellular toxic and soluble Aβ oligomers by activation of autophagy.

The phosphodiesterase 5 inhibitor of the present invention is at least one selected from the group consisting of mirodenafil, sildenafil, vardenafil, tadalafil, udenafil, dasantafil, avanafil; and pharmaceutically acceptable salts, solvates, and hydrates thereof.

The pharmaceutically acceptable salts refer to a formulation of a compound that does not cause serious irritation to an organism to which the compound is administered and does not impair the biological activity and properties of the compound. The pharmaceutically acceptable salts are prepared by conventional methods well known in the art using pharmaceutically acceptable and substantially non-toxic organic and inorganic acids. The acid includes inorganic acids such as hydrochloric acid, bromic acid, sulfuric acid, nitric acid and phosphoric acid; and organic acids such as sulfonic acids, such as methanesulfonic acid, ethanesulfonic acid, and p-toluenesulfonic acid, tartaric acid, formic acid, citric acid, acetic acid, trichloroacetic acid, trifluoroacetic acid, capric acid, isobutane acid, malonic acid, succinic acid, phthalic acid, gluconic acid, benzoic acid, lactic acid, fumaric acid, maleic acid, and salicylic acid. In addition, the compound of the present invention may be reacted with a base to form ammonium salts; alkali metal salts such as sodium or potassium salts; salts such as alkali earth metal salts such as calcium or magnesium salts; salts of organic bases such as dicyclohexylamine, N-methyl-D-glucamine, tris (hydroxymethyl) methylamine; and amino acid salts such as arginine and lysine.

According to one embodiment of the present invention, examples of the pharmaceutically acceptable salts may be mirodenafil hydrochloride, sildenafil citrate, or vardenafil hydrochloride.

The hydrate refers to a compound of the present invention comprising a stoichiometric or non-stoichiometric amount of water bound by a non-covalent intermolecular force, or a salt thereof.

The solvate refers to a compound of the present invention comprising a stoichiometric or non-stoichiometric amount of a solvent bound by a non-covalent intermolecular force, or a salt thereof. Preferred solvents therefor are those that are volatile, non-toxic, and/or suitable for administration to humans.

The acetylcholinesterase inhibitor of the present invention is at least one selected from the group consisting of donepezil, rivastigmine, galantamine; and pharmaceutically acceptable salts, solvates, and hydrates thereof.

More preferably, the phosphodiesterase 5 inhibitor is selected from the group among mirodenafil, pharmaceutically acceptable salts, solvates, hydrates or a mixture thereof, and the acetylcholinesterase inhibitor is selected from the group among donepezil, galantamine, rivastigmine, pharmaceutically acceptable salts, solvates, hydrates or a mixture thereof.

The pharmaceutical composition of the present invention may be administered orally or parenterally.

According to an embodiment of the present invention, the pharmaceutical composition of the present invention is orally administered to a subject or non-orally administered to a site other than the head. In other words, the composition of the present invention may exhibit the effect intended in the present invention even when not directly administered to the brain tissue, the body tissue surrounding the brain tissue (e.g., scalp), and a site adjacent thereto. In one specific example, the non-oral administration is subcutaneous administration, intravenous administration, intraperitoneal injection, transdermal administration, or intramuscular administration, and in another specific example, it is subcutaneous administration, intravenous administration, or intramuscular administration.

The pharmaceutically acceptable carriers comprised in the pharmaceutical composition of the present invention are those commonly used for formulation and include lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate and mineral oil, but are not limited thereto. The pharmaceutical composition of the present invention may further include a lubricant, a wetting agent, a sweetening agent, a flavoring agent, an emulsifying agent, a suspending agent, and a preservative, in addition to the above components. The suitable pharmaceutically acceptable carriers and agents are described in detail in Remington's Pharmaceutical Sciences (19th ed., 1995).

The pharmaceutical composition of the present invention may be prepared in a unit dose form by formulating using a pharmaceutically acceptable carrier and/or excipient, or may be prepared by internalizing in a multi-dose container according to a method that can be easily carried out by a person skilled in the art to which the invention appertains. In this case, the formulation may be in the form of a solution, suspension, or emulsion in an oil or aqueous medium, or in the form of an extract, powder, granule, tablet, film, or capsule, and may further comprise a dispersing agent or a stabilizing agent.

In a certain embodiment, the compositions of the present invention provide synergistic effects on inhibition of proinflammatory factors to provide reduction of neuroinflammation.

In another embodiment, the compositions of the present invention provide synergistic effects on reduction of Aβ42 accumulation, to prevent and/or treat dementia through reduction of amyloid beta by combined use of an phosphodiesterase 5 (PDE5 inhibitor) and an acetylcholinesterase inhibitor.

### EXAMPLES

Hereafter, a more detailed description will be made using the below embodiments. However, these embodiments are only for illustrating the present invention, and the scope of the present invention is not limited by these embodiments.

### Experimental Example 1. Culture Method of IMG Cells

The IMG cell, mouse microglia cell line used in the experiment, and it was cultured in a CO₂ incubator (311-TIF, Thermo Fisher Scientific Forma, MA, USA) under the conditions of 37° C. and 5% CO2 using a DMEM Complete Medium (HyClone) containing 10% fetal bovine serum (FBS; Australian Orgin, HyClone, Logan, UT, USA) and 1% penicillin/streptomycin (P/S; HyClone).

2×10⁵ cells were seeded in each 6-well plate's well and were incubated for 24 hours in the above condition.

After 24 hour incubation, 100 ng/ml of LPS and the drugs, AR1001 and AchEIs (Donepezil, Rivastigmine, Galantamine), were treated in the concentration of 2, 10, 20 μM individually or combined.

### Experimental Example 2. RNA Extraction and cDNA Synthesis

Scrape the cells in the well using a scraper, put 2 mL of the culture solution in a 15 mL conical tube, centrifuge at 3,000 RPM for 5 minutes, discard the culture solution except for the pellets, and treat each cell with 1 mL of Trizol.

Transfer to 1.5 mL e-tube, add 0.2 mL of Chloroform, vortex for 1 minute, and store at room temperature for 2 minutes.

After centrifugation at 12,000 g, 4° C. for 10 minutes, 500 μL of the supernatant was separated and 500 μL of Iso-propanol was added to the separated supernatant and was put at room temperature for 10 minutes, and then centrifuged at 12,000 g, 4° C. for 10 minutes.

The supernatant was discarded and the pellet was washed with 75% EtOH twice.

EtOH was discarded all, and the RNA pellet was dried and dissolved in 10 μL DEPC Treated Water.

cDNA Synthesis was done following the PrimeScript™ II 1st strand cDNA Synthesis Kit (Takara).

### Experimental Example 3. Real Time RT-qPCR for Pro-Inflammatory Cytokines

Real Time RT-qPCR was performed using the Quant Studio 5 (Applied biosystems). The primer sequence for IL1beta forward primer was 5′-AGCTTCAGGCAGGCAGTATC-3′ (SEQ ID NO:1); IL1 beta reverse primer was 5′-AAGGTCCACGGGAAAGACAC-3′ (SEQ ID NO:2); TNFalpha forward primer was 5′-AAATGGCCTCCCTCTCATCAG-3′ (SEQ ID NO: 3); TNFalpha reverse primer was 5′-GTCACTCGAATTTTGAGAAGATGATC-3′ (SEQ ID NO:4); beta actin forward primer was 5′-CGTGCGTGACATCAAAGAGAA-3′ (SEQ ID NO:5); beta actin reverse primer was 5′-TGGATGCCACAGGATTCCAT-3′ (SEQ ID NO:6). SYBR Green PCR Master Mix (ThermoFisher) was used for the polymerase.

### Experimental Example 4. Results of Measuring the Pro-Inflammatory Cytokines Decrease Rate

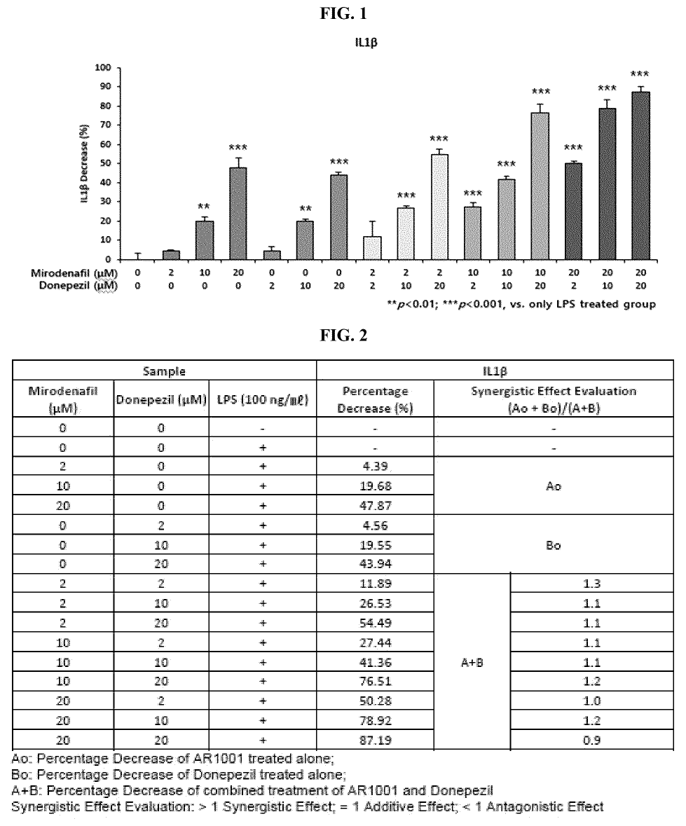

FIG. 1 shows the results of an experiment for checking whether the combined use of mirodenafil and donepezil in the present invention has a synergistic effect on IL-1β.

In FIG. 1, AR1001 refers to mirodenafil, and looking at FIG. 2, the IL-1β reduction rate of 11.89% for a combined treatment of 2 μM of mirodenafil and 2 μM of donepezil; the IL-1β reduction rate of 26.53% for a combined treatment of 2 μM of mirodenafil and 10 μM of donepezil; the IL-1β reduction rate of 54.49% for a combined treatment of 2 μM of mirodenafil and 20 μM of donepezil; and the IL-1β reduction rate of 27.44% for a combined treatment of 10 μM of mirodenafil and 2 μM of donepezil; and the IL-1β reduction rate of 41.36% for a combined treatment of 10 μM of mirodenafil and 10 μM of donepezil; and the IL-1β reduction rate of 76.51% for a combined treatment of 10 μM of mirodenafil and 20 μM of donepezil; and the IL-1β reduction rate of 50.28% for a combined treatment of 20 μM of mirodenafil and 2 μM of donepezil; and the IL-1β reduction rate of 78.92% for a combined treatment of 20 μM of mirodenafil and 10 μM of donepezil; were significantly higher than the sum of the increase rates A and B for a treatment of mirodenafil or donepezil alone, which confirmed that an effect beyond the additive effect can be recognized.

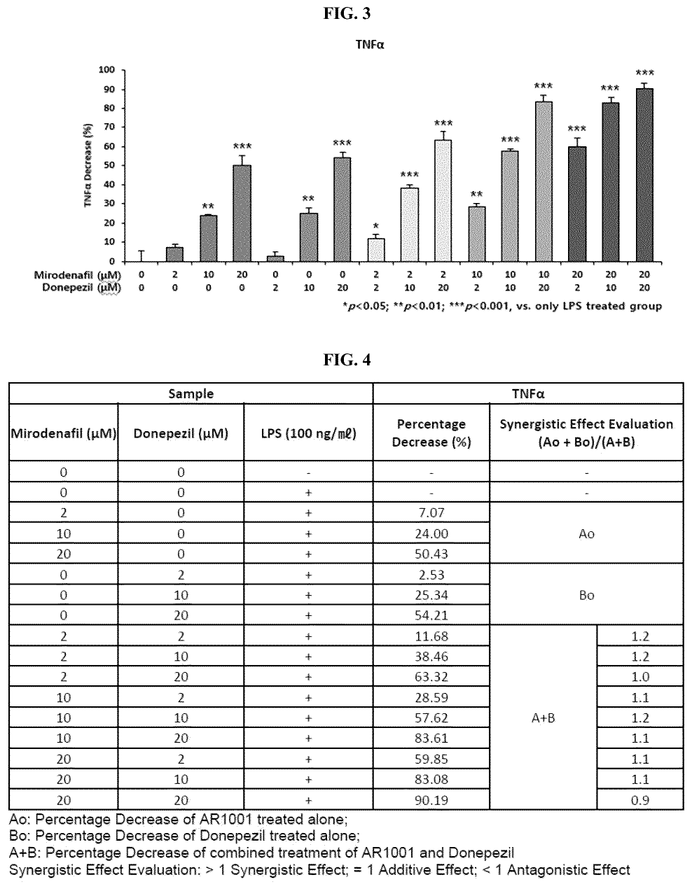

FIG. 3 shows the results of an experiment for checking whether the combined use of mirodenafil and donepezil in the present invention has a synergistic effect on TNF-α.

In FIG. 3, AR1001 refers to mirodenafil, and looking at FIG. 4, the TNF-α reduction rate of 11.68% for a combined treatment of 2 μM of mirodenafil and 2 μM of donepezil; the TNF-α reduction rate of 38.46% for a combined treatment of 2 μM of mirodenafil and 10 μM of donepezil; and the TNF-α reduction rate of 28.59% for a combined treatment of 10 μM of mirodenafil and 2 μM of donepezil; and the TNF-α reduction rate of 57.62% for a combined treatment of 10 μM of mirodenafil and 10 μM of donepezil; and the TNF-α reduction rate of 83.61% for a combined treatment of 10 μM of mirodenafil and 20 μM of donepezil; and the TNF-α reduction rate of 59.85% for a combined treatment of 20 μM of mirodenafil and 2 μM of donepezil; and the TNF-α reduction rate of 83.08% for a combined treatment of 20 μM of mirodenafil and 10 μM of donepezil; were significantly higher than the sum of the increase rates A and B for a treatment of mirodenafil or donepezil alone, which confirmed that an effect beyond the additive effect can be recognized.

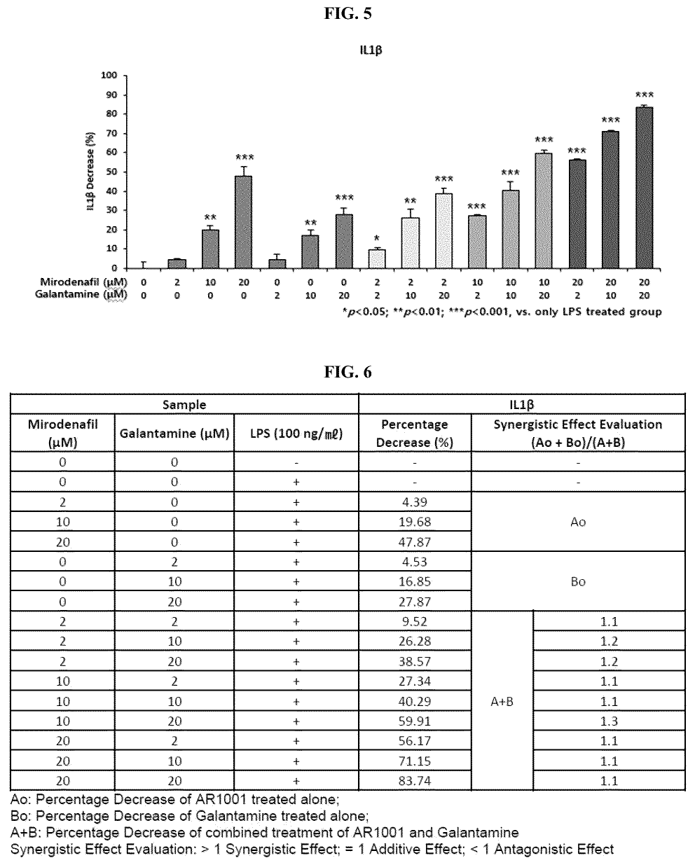

FIG. 5 shows the results of an experiment for checking whether the combined use of mirodenafil and galantamine in the present invention has a synergistic effect on IL-1β.

In FIG. 5, AR1001 refers to mirodenafil, and looking at FIG. 6, the IL-1β reduction rate of 9.52% for a combined treatment of 2 μM of mirodenafil and 2 μM of galantamine; the IL-1β reduction rate of 26.28% for a combined treatment of 2 μM of mirodenafil and 10 μM of galantamine; the IL-1β reduction rate of 38.57% for a combined treatment of 2 μM of mirodenafil and 20 μM of galantamine; and the IL-1β reduction rate of 27.34% for a combined treatment of 10 μM of mirodenafil and 2 μM of galantamine; and the IL-1β reduction rate of 40.29% for a combined treatment of 10 μM of mirodenafil and 10 μM of galantamine; and the IL-1β reduction rate of 59.91% for a combined treatment of 10 μM of mirodenafil and 20 μM of galantamine; and the IL-1β reduction rate of 56.17% for a combined treatment of 20 μM of mirodenafil and 2 μM of galantamine; and the IL-1β reduction rate of 71.15% for a combined treatment of 20 μM of mirodenafil and 10 μM of galantamine; and the IL-1β reduction rate of 83.74% for a combined treatment of 20 μM of mirodenafil and 20 μM were significantly higher than the sum of the increase rates A and B for a treatment of mirodenafil or galantamine alone, which confirmed that an effect beyond the additive effect can be recognized.

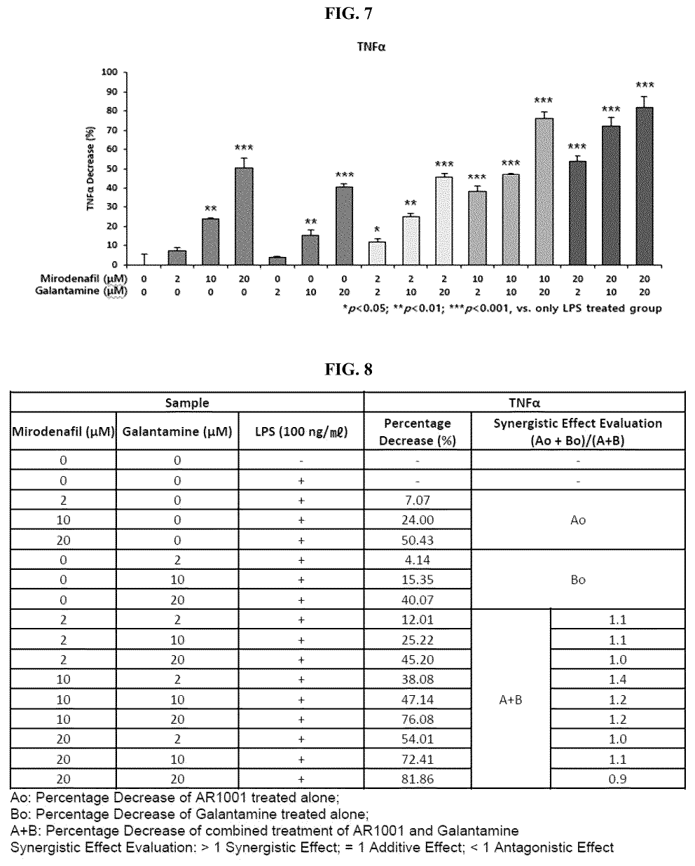

FIG. 7 shows the results of an experiment for checking whether the combined use of mirodenafil and galantamine in the present invention has a synergistic effect on TNF-α.

In FIG. 7, AR1001 refers to mirodenafil, and looking at FIG. 8, the TNF-α reduction rate of 12.01% for a combined treatment of 2 μM of mirodenafil and 2 μM of galantamine; the TNF-α reduction rate of 25.22% for a combined treatment of 2 μM of mirodenafil and 10 μM of galantamine; and the TNF-α reduction rate of 38.08% for a combined treatment of 10 μM of mirodenafil and 2 μM of galantamine; and the TNF-α reduction rate of 47.14% for a combined treatment of 10 μM of mirodenafil and 10 μM of galantamine; and the TNF-α reduction rate of 76.08% for a combined treatment of 10 μM of mirodenafil and 20 μM of galantamine; and the TNF-α reduction rate of 72.41% for a combined treatment of 20 μM of mirodenafil and 10 μM of galantamine; were significantly higher than the sum of the increase rates A and B for a treatment of mirodenafil or galantamine alone, which confirmed that an effect beyond the additive effect can be recognized.

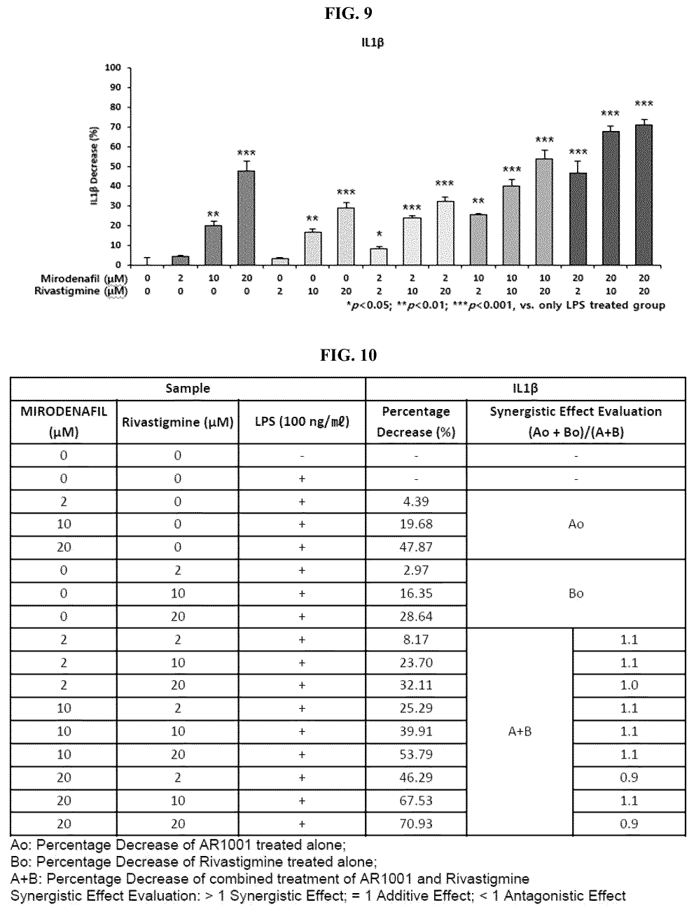

FIG. 9 shows the results of an experiment for checking whether the combined use of mirodenafil and rivastigmine in the present invention has a synergistic effect on IL-1.

In FIG. 9, AR1001 refers to mirodenafil, and looking at FIG. 10, the IL-1β reduction rate of 8.17% for a combined treatment of 2 μM of mirodenafil and 2 μM of rivastigmine; the IL-1β reduction rate of 23.70% for a combined treatment of 2 μM of mirodenafil and 10 μM of rivastigmine; and the IL-1β reduction rate of 25.29% for a combined treatment of 10 μM of mirodenafil and 2 μM of rivastigmine; and the IL-1β reduction rate of 39.91% for a combined treatment of 10 μM of mirodenafil and 10 μM of rivastigmine; and the IL-1β reduction rate of 53.79% for a combined treatment of 10 μM of mirodenafil and 20 μM of rivastigmine; and the IL-1β reduction rate of 67.53% for a combined treatment of 20 μM of mirodenafil and 10 μM of rivastigmine; were significantly higher than the sum of the increase rates A and B for a treatment of mirodenafil or rivastigmine alone, which confirmed that an effect beyond the additive effect can be recognized.

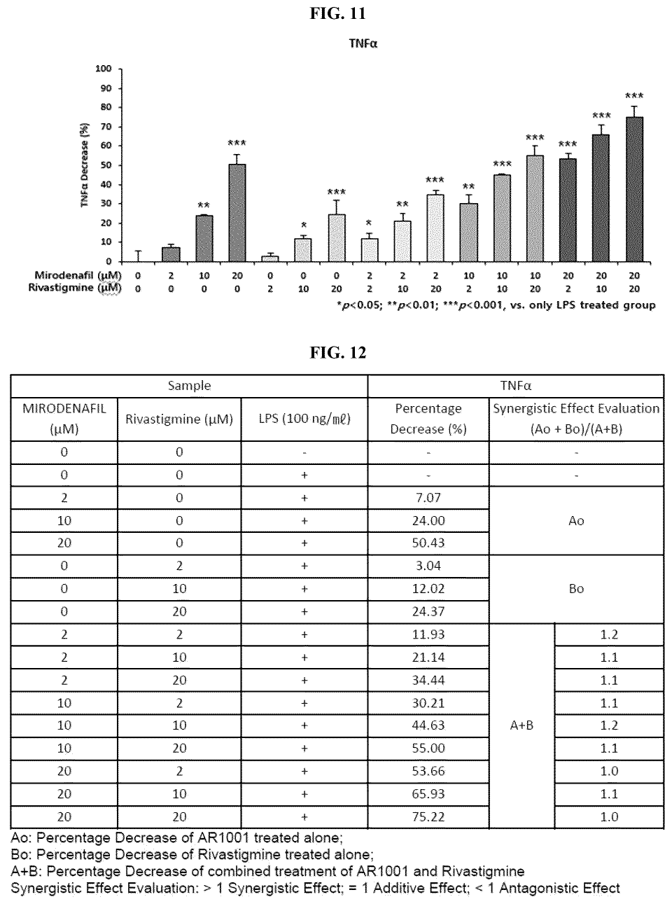

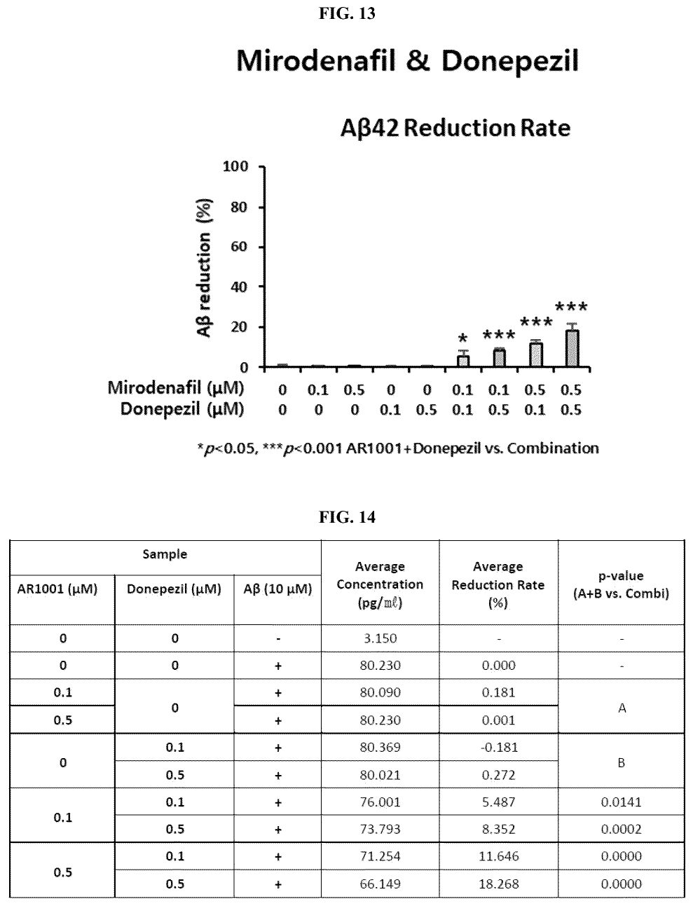

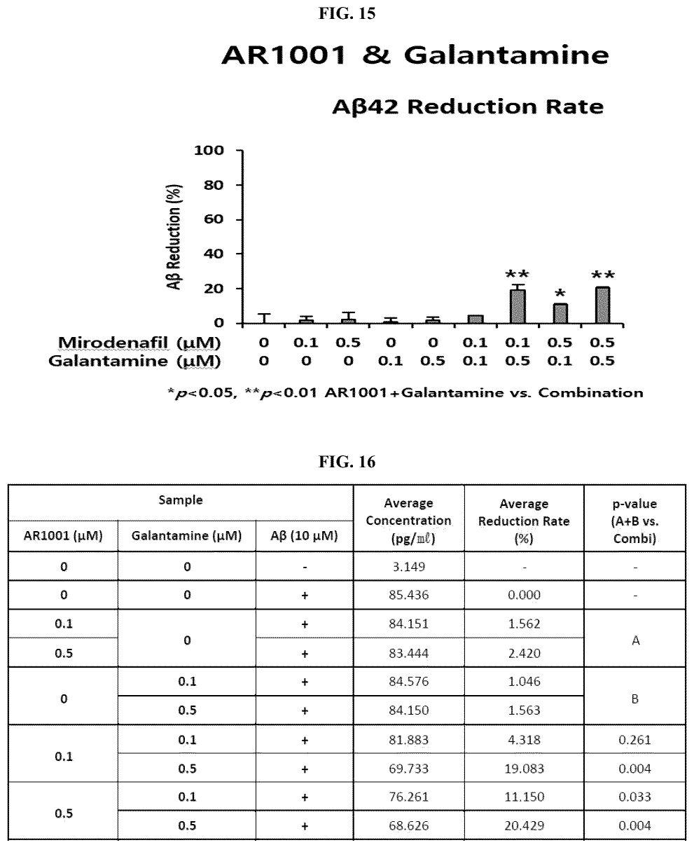

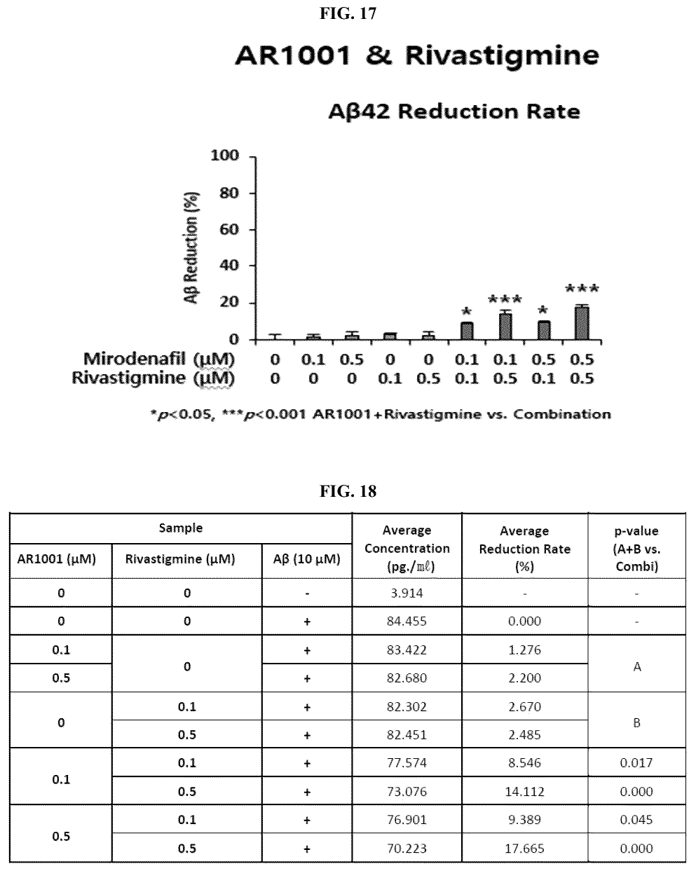

FIG. 11 shows the results of an experiment for checking whether the combined use of mirodenafil and rivastigmine in the present invention has a synergistic effect on TNF-α.

In FIG. 12, AR1001 refers to mirodenafil, and looking at FIG. 8, the TNF-α reduction rate of 11.93% for a combined treatment of 2 μM of mirodenafil and 2 μM of rivastigmine; the TNF-α reduction rate of 21.14% for a combined treatment of 2 μM of mirodenafil and 10 μM of rivastigmine; the TNF-α reduction rate of 34.44% for a combined treatment of 2 μM of mirodenafil and 20 μM of rivastigmine; and the TNF-α reduction rate of 30.21% for a combined treatment of 10 μM of mirodenafil and 2 μM of rivastigmine; and the TNF-α reduction rate of 44.63% for a combined treatment of 10 μM of mirodenafil and 10 μM of rivastigmine; and the TNF-α reduction rate of 55.00% for a combined treatment of 10 μM of mirodenafil and 20 μM of rivastigmine; and the TNF-α reduction rate of 65.93% for a combined treatment of 20 μM of mirodenafil and 10 μM of rivastigmine; were significantly higher than the sum of the increase rates A and B for a treatment of mirodenafil or rivastigmine alone, which confirmed that an effect beyond the additive effect can be recognized.

### Experimental Example 5. Cell Culture

The SH-SY5Y human neuroblastoma cell line used in the experiment was purchased from American Type Culture Collection (ATCC; Manassas, VA, USA), and it was cultured in a CO₂ incubator (311-TIF, Thermo Fisher Scientific Forma, MA, USA) under the conditions of 37° C. and 5% CO2 using a DMEM/F12 Complete Medium (HyClone) containing 10% fetal bovine serum (FBS; Australian Orgin, HyClone, Logan, UT, USA) and 1% penicillin/streptomycin (P/S; HyClone).

### Experimental Example 6. Neuron-Like Differentiation of SH-SY5Y Cells Using all-Trans-Retinoic Acid (RA)

2×104 [sic] cells/well were dispensed into a 96-well plate to evaluate cytotoxicity, and 2×105 [sic] cells were dispensed into a T-25 flask to check neuronal cell death, neuronal inflammatory response, protein expression changes related to neurotransmitters and synaptic plasticity, and activity of acetylcholinesterase (AChE) activity. For cell fixation and stabilization, a DMEM/F12 Complete Medium (HyClone) containing 10% FBS (HyClone) and 1% P/S (HyClone) was used to culture for 24 hours in a CO₂ incubator (Thermo Fisher Scientific Forma) under the conditions of 37° C. and 5% CO₂. 24 hours after cell dispensing, the cell culture medium was removed for neuron-like differentiation and replaced with a DMEM/F12 differentiation medium containing 1% FBS (HyClone), 1% P/S (HyClone), and 10 μM all-trans-retinoic acid (RA; Sigma-Aldrich, St. Louis, MO, USA). On the third day of differentiation, the medium was replaced with a new DMEM/F12 differentiation medium. On the sixth day of differentiation, the medium for the untreated control group was replaced with a new DMEM/F12 differentiation medium, and the sample treated group was replaced by adding a new DMEM/F12 differentiation medium under various conditions.

### Experimental Example 7. Formation and Treatment of Amyloid β(Aβ)1-42

To form Aβ1-42 oligomers, human Aβ1-42 (Abcam, Cambridge, MA, USA) was added to a DMEM/F12 Complete Medium (HyClone) containing 1% FBS (HyClone) and 1% P/S (HyClone) to make 10 μM and left for three hours in a CO₂ incubator (Thermo Fisher Scientific Forma) under the conditions of 37° C. and 5% CO₂ to form Aβ1-42 oligomers.

To check the Aβ1-42 change, the existing cell culture medium was removed from the RA-differentiated SH-SY5Y neuron-like cell, and replaced with a DMEM/F12 Complete Medium (HyClone) containing Aβ1-42 oligomers (10 μM), and cultured for 72 hours in a CO₂ incubator (Thermo Fisher Scientific Forma) under the conditions of 37° C. and 5% CO₂ to induce Aβ1-42 oligomer-induced cell damage.

After 72 hours, the culture medium was removed, and then the DMEM/F12 Complete Medium (HyClone) was treated alone or in combination with mirodenafil, donepezil, galantamine or rivastigmine and cultured for 24 hours in a CO₂ incubator (Thermo Fisher Scientific Forma) under the conditions of 37° C. and 5% CO₂, and then the experiment was carried out.

### Experimental Example 8. Result of Measuring Human Aβ 42 ELISA (Enzyme-Linked Immunosorbent Assay)

To measure the amount of Aβ42 in the cells (pg/mL), the cells were recovered and treated with a Cell Lysis buffer. Thereafter, after 10 minutes of centrifugation at 14,000 rpm at 4° C., the supernatant is transferred, and protein is recovered. The amount of protein was quantified using the Pierce™ BCA Protein Assay Kit (Thermo Fisher Scientific). Then, the Human Aβ42 ELISA Kit (Invitrogen) was used to measure the amount of Aβ42 in the cells.

FIG. 14 shows the results of an experiment for checking whether the combined use of mirodenafil and donepezil in the present invention has a synergistic effect on Aβ reduction.

In FIG. 14, AR1001 refers to mirodenafil, and looking at FIG. 14, the Aβ reduction rate of 5.49% for a combined treatment of 0.1 μM of mirodenafil and 0.1 μM of donepezil; the Aβ reduction rate of 8.35% for a combined treatment of 0.1 μM of mirodenafil and 0.5 μM of donepezil; the Aβ reduction rate of 11.65% for a combined treatment of 0.5 μM of mirodenafil and 0.1 μM of donepezil; and the Aβ reduction rate of 18.27% for a combined treatment of 0.5 M of mirodenafil and 0.5 μM of donepezil were significantly higher than the sum of the increase rates A and B for a treatment of mirodenafil or donepezil alone, which confirmed that an effect beyond the additive effect can be recognized.

FIG. 16 shows the results of an experiment for checking whether the combined use of mirodenafil and galantamine in the present invention has a synergistic effect on Aβ reduction.

Looking at FIG. 16, the Aβ reduction rate of 4.32% for a combined treatment of 0.1 M of mirodenafil and 0.1 μM of galantamine; the Aβ reduction rate of 19.08% for a combined treatment of 0.1 μM of mirodenafil and 0.5 μM of galantamine; the Aβ reduction rate of 11.15% for a combined treatment of 0.5 μM of mirodenafil and 0.1 μM of galantamine; and the Aβ reduction rate of 20.43% for a combined treatment of 0.5 μM of mirodenafil and 0.5 μM of galantamine were significantly higher than the sum of the increase rates A and B for a treatment of mirodenafil or galantamine alone, which confirmed that an effect beyond the additive effect can be recognized.

FIG. 18 shows the results of an experiment for checking whether the combined use of mirodenafil and rivastigmine in the present invention has a synergistic effect on Aβ reduction.

Looking at FIG. 18, the Aβ reduction rate of 8.43% for a combined treatment of 0.1 M of mirodenafil and 0.1 μM of rivastigmine; the Aβ reduction rate of 14.88% for a combined treatment of 0.1 μM of mirodenafil and 0.5 μM of rivastigmine; the Aβ reduction rate of 8.94% for a combined treatment of 0.5 μM of mirodenafil and 0.1 μM of rivastigmine; and the Aβ reduction rate of 17.91% for a combined treatment of 0.5 μM of mirodenafil and 0.5 μM of rivastigmine were significantly higher than the sum of the increase rates A and B for a treatment of mirodenafil or rivastigmine alone, which confirmed that an effect beyond the additive effect can be recognized.

The present invention described above is merely illustrative and a person skilled in the art to which the present invention appertains will understand that various modifications and other equivalent embodiments are possible therefrom. Therefore, it will be understood that the present invention is not limited to the form mentioned in the detailed description above. Therefore, the true scope of the technical protection of the present invention should be determined by the technical idea of the appended scope of claims. In addition, it is to be understood that the present invention covers all modifications, equivalents, and substitutions within the spirit and scope of the present invention defined by the appended scope of claims.

Sequence List

| | | SEQ |
|---|---|---|
| | | ID |
| Description | Sequence | NO. |
| IL1beta | 5′-AGCTTCAGGCAGGCAGTATC-3′ | 1 |
| forward primer | | |
| IL1beta | 5′-AAGGTCCACGGGAAAGACAC-3′ | 2 |
| reverse primer | | |
| TNFalpha | 5′-AAATGGCCTCCCTCTCATCAG-3′ | 3 |
| forward primer | | |
| TNFalpha | 5′-GTCACTCGAATTTTGAGAAGATGA | 4 |
| reverse primer | TC-3′ | |
| beta actin | 5′-CGTGCGTGACATCAAAGAGAA-3′ | 5 |
| forward primer | | |
| beta actin | 5′-TGGATGCCACAGGATTCCAT-3′ | 6 |
| reverse primer | | |

1-16. (canceled)

17. A pharmaceutical composition comprising:
at least one phosphodiesterase 5 inhibitor; and
at least one acetylcholinesterase inhibitor as active ingredients, and pharmaceutically acceptable salts, solvates, hydrates of said phosphodiesterase 5 inhibitor and said acetylcholinesterase inhibitor.

18. The composition of claim 17, wherein the phosphodiesterase 5 inhibitor comprises mirodenafil, sildenafil, vardenafil, tadalafil, udenafil, dasantafil, avanafil, and derivatives and mixtures thereof.

19. The composition of claim 18, wherein the phosphodiesterase 5 inhibitor is mirodenafil and derivatives thereof.

20. The composition of claim 17, wherein the acetylcholinesterase inhibitor comprises donepezil, rivastigmine, galantamine, physostigmine, tacrine, metrifonate, phenserine, tolserine, eseroline, huperizine A and B, galangin, cardanol, donepezil-AP2238, donepezil-tacrine, tacrine-ferulic acid hybrid, tacrine-hydroxyquinoline, ladostigil, indenyl derivatives, and mixtures thereof.

21. A method for preventing or treating neuroinflammation comprising:
administering an effective amount of the pharmaceutical composition of claim 17.

22. A method for preventing and/or inhibiting formation and/or accumulation of beta-amyloid comprising:
administering an effective amount of the pharmaceutical composition of claim 17.

23. A method for preventing or treating a neurodegenerative disease comprising:
administering an effective amount of the pharmaceutical composition of claim 17.

24. The method of claim 23, wherein the neurodegenerative disease comprises dementia, Parkinson's disease (PD), Dementia with Lewy body (DLB), Alzheimer's disease (AD), Huntington's disease (HD), Multiple sclerosis (MS), Vascular Dementia (VaD), and a mixed etiologies thereof.

25. A method for preventing or treating Dementia comprising:
administering an effective amount of the pharmaceutical composition of claim 17.

26. A method for inhibiting Aβ Oligomer/Fibril formation by reducing Aβ aggregation comprising:
administering an effective amount of the pharmaceutical composition of claim 17.

27. A method for inhibiting β-Amyloidogenic processing by reducing BACE-1 comprising:
administering an effective amount of the pharmaceutical composition of claim 17.

28. A method for reducing extracellular Aβ monomers, oligomers & Aβ Fibril/Plaque by increasing the cerebral blood flow comprising:
administering an effective amount of the pharmaceutical composition of claim 17.

29. A method for suppressing neuronal cell death, promoting neurogenesis, synaptogenesis and/or angiogenesis by activating NO/cGMP/PKG/CREB Pathway comprising:
administering an effective amount of the pharmaceutical composition of claim 17.

30. A method for restoring synaptic plasticity by activating Wint signaling and/or inhibiting DKK-1 comprising:
administering an effective amount of the pharmaceutical composition of claim 17.

31. A method for inhibiting production of APP and/or reducing Aβ accumulation by suppressing positive feedback loop for Aβ production comprising:
administering an effective amount of the pharmaceutical composition of claim 17.

32. A method for inhibiting formation of Aβ Fibril/plaque by removing intracellular toxic and soluble Aβ oligomers by activating autophagy comprising:
administering an effective amount of the pharmaceutical composition of claim 17.

33. A pharmaceutical composition,
mirodenafil, or pharmaceutically acceptable salts, solvates, hydrates, derivatives thereof; and
at least one of donepezil, galantamine, rivastigmine, or pharmaceutically acceptable salts, solvates, hydrates, derivatives, and/or mixtures thereof.

34. A method for preventing or treating neuroinflammation comprising:
administering an effective amount of the pharmaceutical composition of claim 33.

35. A method for preventing or inhibiting formation and/or accumulation of beta-amyloid comprising:
administering an effective amount of the pharmaceutical composition of claim 33.

36. A method for preventing or treating a neurodegenerative disease comprising:
administering an effective amount of the pharmaceutical composition of claim 33.

37. The method of claim 36, wherein the neurodegenerative disease comprises dementia, Parkinson's disease (PD), Dementia with Lewy body (DLB), Alzheimer's disease (AD), Huntington's disease (HD), Multiple sclerosis (MS), Vascular Dementia (VaD), and a mixed etiologies thereof.

38. A method for preventing or treating Dementia comprising:
administering an effective amount of the pharmaceutical composition of claim 33.

39. A method for inhibiting Aβ Oligomer/Fibril formation by reducing Aβ aggregation comprising:
administering an effective amount of the pharmaceutical composition of claim 33.

40. A method for inhibiting β-Amyloidogenic processing by reducing BACE-1 comprising:
administering an effective amount of the pharmaceutical composition of claim 33.

41. A method for reducing extracellular Aβ monomers, oligomers & Aβ Fibril/Plaque by increasing the cerebral blood flow comprising:
administering an effective amount of a pharmaceutical composition of claim 33.

42. A method for suppressing neuronal cell death, promoting neurogenesis, synaptogenesis and/or angiogenesis by activating NO/cGMP/PKG/CREB Pathway comprising:
administering an effective amount of the pharmaceutical composition of claim 33.

43. A method for restoring synaptic plasticity by activating Wint signaling and/or inhibiting DKK-1 comprising:
administering an effective amount of the pharmaceutical composition of claim 33.

44. A method for inhibiting the production of APP and/or reducing Aβ accumulation by suppressing positive feedback loop for Aβ production comprising:
administering an effective amount of the pharmaceutical composition of claim 33.

45. A method for inhibiting formation of Aβ Fibril/plaque by removing intracellular toxic and soluble Aβ oligomers by activating autophagy comprising:
administering an effective amount of the pharmaceutical composition of claim 33.


SEQUENCE LISTING (6 sequences)

SEQ ID NO: 1 - DNA, 20 bp, Homo sapiens
  source 1..20: mol_type = genomic DNA
agcttcaggcaggcagtatc

SEQ ID NO: 2 - DNA, 20 bp, Homo sapiens
  source 1..20: mol_type = genomic DNA
aaggtccacgggaaagacac

SEQ ID NO: 3 - DNA, 21 bp, Homo sapiens
  source 1..21: mol_type = genomic DNA
aaatggcctccctctcatcag

SEQ ID NO: 4 - DNA, 26 bp, Homo sapiens
  source 1..26: mol_type = genomic DNA
gtcactcgaattttgagaagatgatc

SEQ ID NO: 5 - DNA, 21 bp, Homo sapiens
  source 1..21: mol_type = genomic DNA
cgtgcgtgacatcaaagagaa

SEQ ID NO: 6 - DNA, 20 bp, Homo sapiens
  source 1..20: mol_type = genomic DNA
tggatgccacaggattccat